(19) United States
(12) Patent Application Publication
(10) Pub. No.: US 2007/0130732 A1
(43) Pub. Date: Jun. 14, 2007

(54) Fastening element and article equipped with same

(76) Inventors: Yoshiyuki Matsumura, Kanagawa (JP); Satoshi Goto, Iwate (JP)

Correspondence Address: 3M INNOVATIVE PROPERTIES COMPANY, PO BOX 33427, ST. PAUL, MN, 55133-3427, US

(21) Appl. No.: 10/575,207

(22) PCT Filed: Oct. 6, 2004
(86) PCT No.: PCT/US04/32814

§ 371 Date: Apr. 10, 2006

(30) Foreign Application Priority Data
Oct. 10, 2003 (JP) 2003-352691

(51) Int. Cl. A61F 13/15

(52) U.S. Cl. 24/442; 604/361; 604/387

(57) ABSTRACT

To provide a fastening tape equipped with an extensible material or other fastening elements that are free from excessive extension and contraction of an extensible member, can always keep a suitable degree of extension and contraction, do not therefore generate a disadvantage such as a red mark when they are used for a disposable diaper, and make it possible to easily recognize with eye that the suitable degree of extension and contraction is achieved. In a fastening element for detachably fixing two members, the fastening element is constituted so that it comprises a fastening member having an extensible member incorporating therein an indicator capable of visually showing a degree of extension and contraction.

### TECHNICAL FIELD

This invention relates to a fastening element. More particularly, the invention relates to a fastening element that can detachably fix two members and can drastically improve handling property of a fastening member and other performances by using conjointly an extensible member. The invention relates also to an article equipped with such a fastening element, particularly a disposable diaper.

### BACKGROUND ART

A variety of disposable diapers have been devised and put on the market in the past for babies, inpatients and incontinentia. The disposable diapers that are commercially available can be divided into an underpants type that can be put as such to a wearer and an expansion type (open type) that has an elongated shape and can be put by using a fastening tape after being put to the wearer. The fastening tape uses sometimes a re-peeling type adhesive tape that can be bonded to a diaper main body and can repeat bonding and peeling operations, and typically uses a surface fastener (also called “mechanical fastener”) comprising a combination of hooks and loops.

The open type disposable diaper generally includes an inner sheet made of a liquid permeable inner material, a liquid impermeable back sheet put in such a manner as to cover the inner decoration sheet, a frontal film for reinforcing the back sheet and fixing a fastening tape, arranged at a predetermined portion of the back sheet, and a fastening tape fastened to the frontal film, for completing the shape of the diaper and stably keeping the shape.

In the disposable diapers described above, an extensible member is generally used for the fitting portion of the fastening tape so as to fit to the body of the wearer, particularly to the girth and the crotch (patent reference 1). Although not illustrated, the disposable diaper described in the patent reference 1 includes a liquid permeable surface sheet, a liquid impermeable back sheet and an absorbing member capable of retaining a liquid and sandwiched between these sheets. In this disposable diaper, end portions of the waist belt is put one upon another and the superposed portion is covered with an end portion of the back sheet and is fixed through a male member of the mechanical fastener. The disposable diaper can thus be fitted to the body of the wearer. To fit the diaper to the body of the wearer, a flexible member is provided to the waist portion. Further, an extensible member formed of an extensible cloth is disposed at the proximal end of the waist belt. Because of the extensible member, the waist belt can follow the movement of the wearer, fittability can be further improved, and fixing of the waist belt can be further insured.

Nonetheless, the extensible member used for the disposable diaper involves several problems. For example, the shrinkage force of the extensible member is generally likely to become higher when it is stretched more. When the extensible member is excessively stretched, a load is applied to the girth and the crotch of the wearers inclusive of babies. Even when fittability is improved by providing a flexible member to the waist portion, for example, it is difficult to prevent the occurrence of a red trace of pushing (so-called “red mark”). When the extensible member is not sufficiently stretched, the shrinkage force becomes low and the diaper is fitted loosely. As a result, deviation of the diaper occurs and the occurrence of liquid leak cannot be prevented easily. Still another problem is that because the extensible member can be stretched and contracted arbitrarily, adjustment of the degree of extension and contraction is extremely difficult. In effect, the wearers empirically judge the degree of extension and contraction of the extensible member from the touch feeling they acquire when putting the diaper and from the touch feeling acquired by touching the diaper as a whole or from its appearance after putting. A product having a construction in which a plurality of scales is provided to the diaper main body and the wearer puts the distal end of the fastening tape to an optimum scale in accordance with the age has been proposed. However, this diaper does not provide the fundamental solution for the problems described above.

### DISCLOSURE OF THE INVENTION

It is therefore an object of the invention to provide a fastening tape equipped with an extensible member or other fastening elements that can solve the problems of the prior art described above, prevent excessive and insufficient stretching of the extensible member and can moreover secure an appropriate degree of extension and contraction.

It is another object of the invention to provide a fastening tape equipped with an extensible member or other fastening elements that make it possible to easily recognize whether or not an appropriate degree of extension and contraction is achieved.

It is still another object of the invention to provide an article that fully exhibits the effect of use when the fastening element of the invention is used.

Especially, it is an object of the invention to provide a disposable diaper that can be put easily to babies and other wearers without involving problems such as a red mark.

The above and other objects of the invention will be readily understood from the following detailed explanation thereof.

According to an aspect of the invention, there is provided a fastening element for detachably fixing two members, in which the fastening element comprises a fastening member having an extensible member incorporating therein an indicator capable of visually showing a degree of extension and contraction.

According to another aspect of the invention, there is provided an article comprising a fastening element of the invention.

The article in the invention is preferably a disposable diaper having the fastening element according to the invention.

The invention can provide a fastening tape equipped with an extensible member or other fastening elements that prevent excessive and insufficient stretching of the extensible member but can always secure an appropriate degree of extension and contraction. Because the degree of extension and contraction of the extensible member is appropriate, the causes of the occurrence of the red mark and liquid leak can be eliminated when the fastening element is used for the disposable diaper, for example.

When the fastening element of the invention is employed, it is possible to easily recognize whether or not an appropriate degree of extension and contraction is achieved. Therefore, the fastening element can be advantageously used for the disposable diaper, or the like.

The disposable diaper according to the invention can be easily put to babies and other wearers without involving drawbacks such as the red mark.

In addition, since an indicator was put to a portion of the disposable diaper such as tab, ear or belt, the utilization value of which portion has not particularly been noticed in the past, it becomes possible to improve the appearance and design of the diaper and in the case of a baby, the putting operation can be conducted while looking at the indicator and talking with the baby. Therefore, the putting work can be made pleasantly and easily.

The fastening element, and the articles having the fastening element, of the invention, typically a disposable diaper, can be advantageously carried out in various embodiments. Preferred embodiments of the invention will be hereinafter explained with reference to the accompanying drawings.

### BRIEF DESCRIPTION OF THE DRAWINGS

FIG. 1 is a perspective view when a fastening element according to the invention is used for a disposable diaper.

FIG. 2 is an expanded view of the disposable diaper shown in FIG. 1.

FIG. 3 is a sectional view of the fastening element used for the disposable diaper shown in FIG. 1.

FIGS. 4A, 4B and 4C are plan views showing an indicator function of an extensible member assembled into the fastening element used for the disposable diaper shown in FIG. 1.

FIG. 5 is an expanded view (part) showing another example where the fastening element according to the invention is used for a disposable diaper.

FIG. 6 is an expanded view (part) showing still another example where the fastening element according to the invention is used for the disposable diaper.

FIG. 7 is a sectional view showing another example of the extensible member that can be used for the fastening element according to the invention.

FIGS. 8A, 8B and 8C are plan views showing an indicator function of the extensible member shown in FIG. 7.

FIGS. 9A and 9B are sectional views showing still another example of the extensible member that can be used for the fastening element according to the invention.

### DETAILED DESCRIPTION

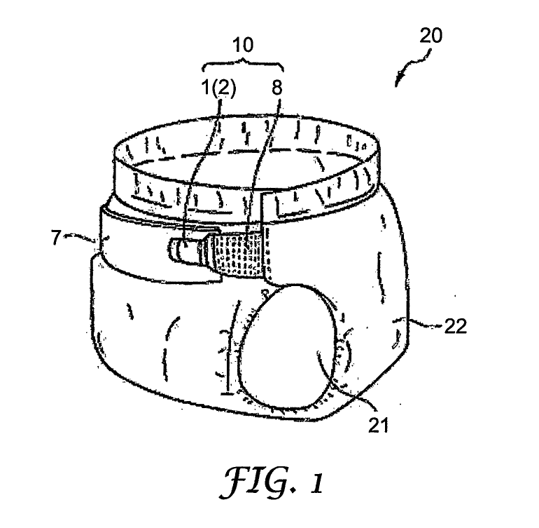

FIG. 1 is a perspective view showing a mode of use of an open type disposable diaper according to a preferred embodiment of the invention that uses a mechanical fastener as a typical example of the fastening element of the invention for fastening a diaper. FIG. 2 is an expanded view of the disposable diaper. It is to be noted prior to the detailed explanation of the embodiment shown in the drawings that the feature of the invention resides particularly in the construction of the fastening element and consequently, the shape of the diaper main body and its construction are not limited to those shown in the drawings but can employ various other shapes and constructions that are conventional in this technical field.

The construction of the open type disposable diaper 20 and its shape are diversified as is well known from various literatures. The disposable diaper 20 generally comprises an inner sheet 21 made of a liquid permeable inner material (non-woven fabric, for example), a liquid impermeable back sheet 22 put on the outside of the inner decoration sheet 21 in such a manner as to wrap the sheet 21, and a liquid absorbing material (not shown) sandwiched between the inner sheet 21 and the back sheet 22, as illustrated. A mechanical fastener (female type) 7 is fitted to a front surface portion (first waist portion) 23 of the back sheet 22 of the diaper 20. A fastening element 10 according to the invention is fitted to a second waist portion 24 on the back sheet 22 of the diaper 20 that forms the front and the back in cooperation with the first waist portion 23 when the diaper is put on a wearer of the diaper. According to the invention, the fastening element 10 comprises a fastening member (male type mechanical fastener in the illustrated example) 2 and an extensible member 8 that can be extended and contracted freely. The distal end of the fastening element 10 is anchored to another fastening member (male type mechanical fastener in the illustrated example) 7 that is used as a pair with the fastening member 2. This distal end is a free end.

As shown in FIG. 2, the disposable diaper is an elongate article having a size and a shape capable of conforming a body shape of the crotch of the wearer wearing the diaper and is compactly folded when not used. When the diaper is used, the folded diaper 20 is expanded and put to the crotch of the wearer. Next, the mechanical fastener 2 of the fastening element 10 having the extensible member 8 is anchored and fixed to the mechanical fastener 7. The disposable diaper 20 shown in FIG. 1 is thus obtained.

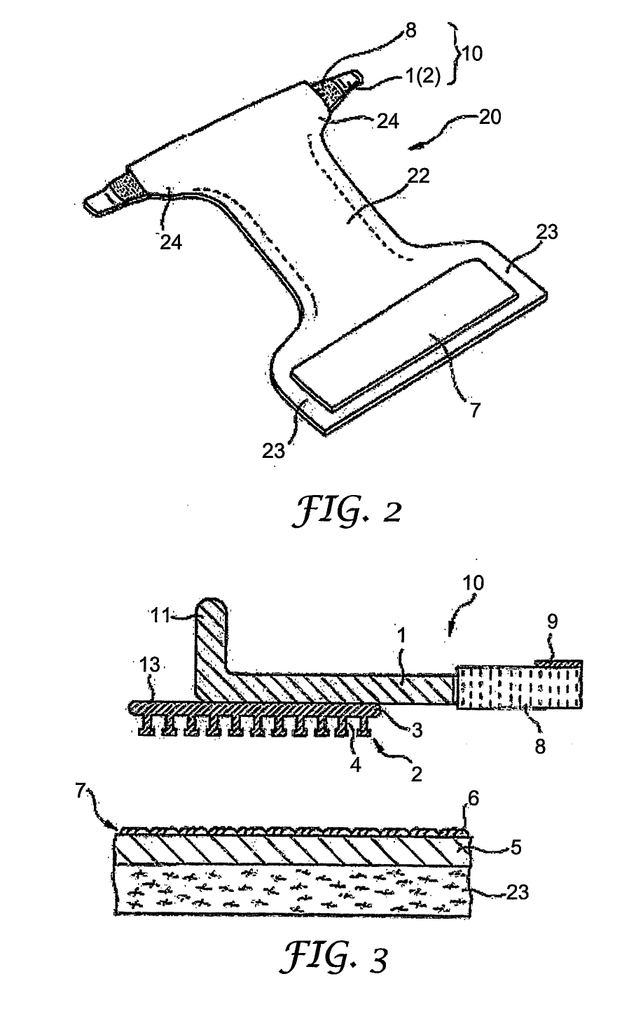

The mechanical fastener used as the fastening member will be then explained. The mechanical fastener (female type) 7 is a loop type clasp. The loop type clasp 7 comprises a base 5 and a loop 6 applied to the base 5 as will be hereinafter explained with reference to FIG. 3. On the other hand, the mechanical fastener (male type) 2 is a hook type clasp. The hook type clasp 2 comprises a hook support 3 and a hook 4 as will be likewise explained hereinafter with reference to FIG. 3. The base 1 of the hook type clasp 2 is unitarily bonded to the extensible member 8. The extensible member 8 is fitted to the second waist portion 24 of the diaper. The distal end portion of the base 1 branches from the free end of the hook type clasp 2 and forms a tab 11 for picking. The mechanical fastener shown in the drawing completes a mechanical fastening state through the engagement between the hook and the loop but the fastening state may well be completed by other mechanisms, whenever necessary. An adhesive tape and a frontal sheet each designed for fastening may be used in place of the hook and the loop, for example.

Next, various members used for the disposable diaper according to the invention will be explained respectively.

In the disposable diaper according to the invention, its main body portion preferably comprises a back sheet and an inner sheet. The back sheet can be formed of a liquid impermeable material conventionally used for the disposable diaper such as an ordinary polyethylene film, a porous polyethylene film, or the like. The thickness of the back sheet can be arbitrarily changed in accordance with the specification of the diaper but is generally within the range of about 5 to 60 μm. The surface of the back sheet may or may not be colored, but is preferably colored to a pale color such as a light blue color or pink in consideration of the feeling of the person wearing the diaper. In the case of the disposal diaper for babies, in particular, pictures of animals, fishes, flowers, characters of popular cartoons, and so forth, are preferably printed.

The inner sheet can be formed of a liquid impermeable material used conventionally for the disposable diaper such as a synthetic fiber including polypropylene, polyethylene, polyester, etc, natural fibers and woven fabric, non-woven fabric and porous sheet of their mixed fibers. The thickness of the inner sheet can be arbitrarily changed in accordance with the specification of the diaper but is generally within the range of about 5 to 60 μm.

FIG. 3 is a sectional view illustrating the fastening element used in the disposable diaper shown in FIG. 1 and the mode of its use. The illustrated fastening element 10 comprises a fastening member 2 called also a “hook type clasp” and an extensible member 8, and is designed so that the hook type clasp 2 is anchored to a loop type clasp 7 to complete the mechanical fastening state. The technology for completing a mechanical fastener by the combination of the loop type clasp and the hook type clasp is well known, and the applicant of the invention has already filed various patent applications.

The fastening element 10 shown in FIG. 3 basically comprises a base 1 and a hook type clasp 2 unitarily combined with each other, and has its features in that the unitary bonding state is released at the distal end, that is, the members are branched or separated at the distal end, and that the branch portion (particularly called hereby “free end”) 11 of the base 1 from the hook type clasp 2 has a size such that when the engagement of the hook type clasp 2 with the loop type clasp 7 is released after it is anchored to the loop type clasp, the free end 11 can form a tab for picking. The size of the tab for picking 11 can be changed in various ways in accordance with the size of the fastening element 10 but is generally within the range of about 10 to 50 mm in width and about 3 to 30 mm in length. The thickness of the base 1 can be changed in accordance with the thickness of the base 1. The tab 11 can be formed by using various methods in an arbitrary stage before, during or after the production of the fastening element 10. When an adhesive layer is disposed on the base 1 supporting the hook type clasp 2, for example, the tab 11 can be formed by masking a part of the adhesive layer with a film of biaxially oriented polypropylene (BOPP) or polyethylene terephthalate (PET), or by folding back the base.

The fastening element according to the invention is characterized in that it comprises an extensible member incorporating therein an indicator capable of visually showing a degree of extension and contraction, in combination with the fastening member. Here, the fastening member and the extensible member are preferably constituted into a unitary structure lest they separate from each other under the ordinary condition of use of the fastening element. The unifying form can be arbitrarily changed, too, in accordance with the specification of the fastening element. For example, one fastening member and one extensible member may be integrated adjacent to each other, or the extensible member may be interposed between two fastening members for unifying. Alternatively, the fastening member may be selectively applied to a part of the extensible member and may be then integrated. The unifying technology of the fastening member and the extensible member is not limited, in particular, and includes, for example, bonding by use of glue, thermal fusion, ultrasonic heating, etc, integral shaping, sewing and mechanical fixing using a stapler.

The extensible member can be formed of various extensible materials capable of freely extending and contracting. Examples of the extensible material suitable for executing the invention, though not restrictive, include elastomer films capable of flexibly extending and contracting or their laminate or composite bodies. Examples of such flexible materials are described in Japanese Examined Patent Publication (Kokoku) No. 6-94188 and U.S. Pat. Nos. 5,691,034 and 5,501,679. It is also possible to use cloths of an extensible sheet either individually or in combination as the extensible material. It is possible to use, as the extensible sheet, the sheets of natural rubber, isoprene rubber, 1,2-polybutadiene, styrene-propylene rubber, urethane rubber and the sheets of various thermoplastic elastomers such as urethane type elastomers, styrene type elastomers and olefin type elastomers. Various fiber materials having extensibility such as woven fabrics and non-woven fabrics can be used as the extensible cloths. The fiber includes synthetic fibers such as polyethylene, polypropylene, polyester and polyamide, and natural fibers such as cotton and rayon. These extensible materials may be used either individually or in combination such as a laminate body, for example. The extensible member is generally used in the form of sheet or tape. The thickness of the extensible member can be changed in a board range but is generally within the range of about 30 to 1,000 mm.

The indicator incorporated in the extensible member of the invention is constituted by lines, patterns, numerals, symbols, illustrations, pictures, photos, color charts, color codes or their arbitrary combinations so that it can be readily recognized. When the fastening element is used for the disposable diaper for babies, for example, the signal can be preferably constituted by pictures or photos of animals, fishes, flowers, popular characters of cartoons and animations that invite the interest of the babies.

The indicator can be imparted to the extensible member through various means but is generally and conveniently imparted by use of printing methods or transfer methods. Printing, for example, can be made through screen-printing, gravure printing and offset printing. Conventional ink such as “Lamic (trade name)” and “Hi-lamic (trade name)” of Dainichiseika Co., Ltd. and “Lamistar (trade name)” and “LP Hi-super (trade name)” of Toyo Ink Co., Ltd. can be used for printing. A sheet having the indicator may be produced separately and may be then bonded to the extensible member. Alternatively, a sheet having the indicator and the extensible member may be simultaneously laminated.

The indicator can be incorporated in various forms into the extensible member. For example, the indicator can be incorporated as a print pattern printed at least partially to the surface of, or into, the extensible member. When the fastening element is used, the extensible member undergoes extension and contraction, and the print pattern can change in accordance with this extension and contraction. The degree of extension and contraction can be readily recognized from the degree of this deformation. Because the indicator deforms, the babies, in particular, watch the mode of change with interest. Therefore, the disposable diaper can be smoothly put.

Alternatively, it is possible to constitute the indicator by a color chart printed at least partially to the surface of the extensible member. The color arrangement can be employed for the color chart so as to acquire various color changes. Preferably, however, the color chart is constituted in such a manner as to form color regions of two or more colors that change regularly in a predetermined direction. To make the color change more clearly recognizable, the color chart is preferably covered with a cover or sheet having a window opening at its substantial center portion. The cover or sheet used herein is preferably made of a material of the type in which the color chart as its foundation cannot be substantially seen through.

The indicator may be formed of a colored porous sheet that is applied at least partially to the surface of the extensible member. The porous sheet displays the color as colored before stretching. When the porous sheet is stretched upon stretching of the extensible member, however, the color of the sheet can vary in accordance with extension and contraction of the extensible member with the increases of voids of the porous sheet. The degree of extraction and contraction of the extensible member can be accurately recognized from this color change condition. In the case of a porous sheet colored to blue, for example, white increases with extension of the extensible member.

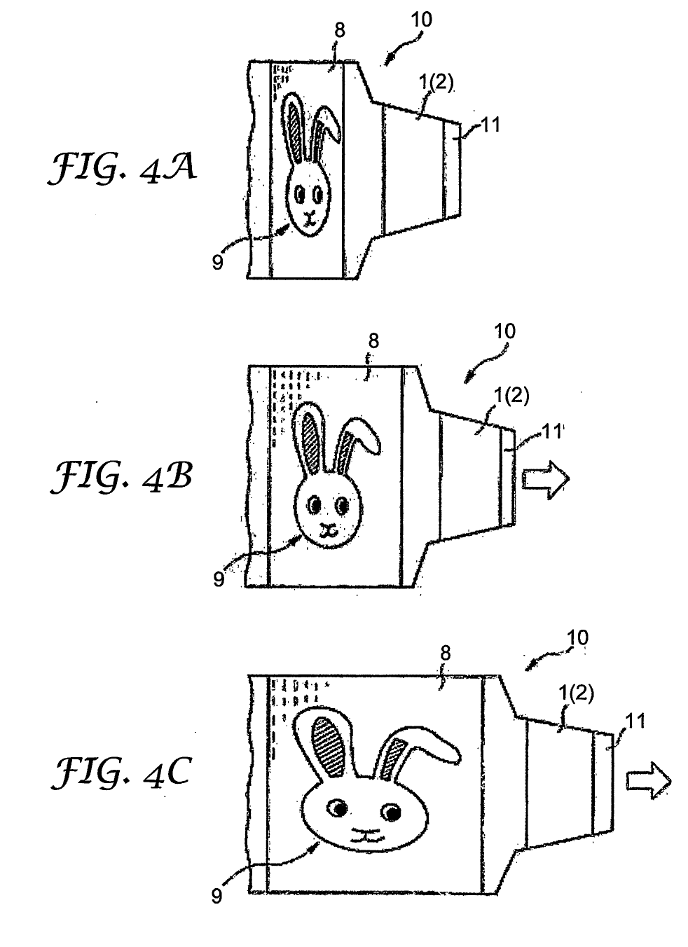

Referring again to FIG. 3, the extensible member 8 is unitarily fitted to the distal end of the hook type clasp 2 (base 1) in the case of the fastening element shown in the drawing. This extensible member 8 is a “Sample B” described in Example 3 of Japanese National Publication (Kohyo) No. 5-501386, and a picture 9 of a rabbit is printed to its surface as shown in FIG. 4. The picture 9 of the rabbit is printed using a gravure printing as the printing method and “Lamic F220 (product name)” of Dainichiseika Co., Ltd. as the printing ink. The proximal end portion of the fastening element 10 (extensible member 8) is fixed through heat seal to the second waist portion 24 of the disposable diaper 20 as shown in FIGS. 1 and 2. Incidentally, when the fastening element 10 is used for the disposable diaper, the fastening element 10 is generally and preferably fixed to and used at the tab, ear or belt of the disposable diaper though it can be fitted to various positions.

The loop type clasp used in combination with the hook type clasp when executing the invention is not particularly limited so long as its loop is adaptable to the hook of the hook type clasp and it does not exert adverse influences on the intended object. The explanation will be given with reference to FIG. 3. The loop type clasp 7 preferably comprises a base 5 and a loop 6 unitarily bonded to the surface of the base 5. The loop 6 can be formed of various materials in a customary manner but is preferably formed of a fiber material capable of effectively entangling with the hook 4 of the hook material 2. The fiber material can be used in the forms such as a woven fabric, a non-woven fabric, a knitted fabric and so forth, in accordance with the shape of the desired loop. Fibers partially bonded onto a film in such a manner that bundles of fibers form the loop shape can be advantageously used as the loop.

FIG. 4 is a plan view showing the display function of the extensible member assembled into the fastening element used in the disposable diaper shown in FIG. 1. FIG. 4(A) shows the state of the fastening element before use. The fastening element 10 includes the extensible member 8 attached on one of the end faces thereof to the second waist portion (not shown) of the disposable diaper and the hook type clasp 2 fitted unitarily on the extension of the extensible member 8 as explained already with reference to FIG. 3. Incidentally, the hook portion cannot be seen in the drawing because it exists on the back, but the base 1 of the hook type clasp 2 can be seen. A face 9 of a rabbit is printed on the surface of the extensible member 8. The face 9 of the rabbit is printed under the state where it is somewhat compressed in the transverse direction so that the face 9 can be seen as a natural face when the extensible member 8 is appropriately extended and contracted. Incidentally, the face 9 of the rabbit is shown printed on the uppermost surface of the extensible member 8 but a different design effect can be obtained when a transparent or translucent cover or sheet is put on the face 9.

To put the disposable diaper, the tab 11 formed on the end face of the fastening element 10 is picked and pulled in the direction of the arrow as shown in FIG. 4(B). As the extensible member 8 is stretched, the face 9 of the rabbit can be seen as a natural face. Such a face represents that extension and contraction of the extensible member 8 is made appropriately. Therefore, the extension and contraction of the extensible member 8 is stopped and the hook type clasp 2 is anchored to the loop type clasp 7 (see FIG. 3) to fix them with each other. Though the face 9 of the rabbit is used as the indicator in the drawing, it will be obvious to those skilled in the art that other pictures and image patterns can also be the scale for clearly representing suitable extension and contraction of the extensible member 8.

Incidentally, when the extensible member 8 is excessively stretched, this excessive stretch can be known from deformation of the face 9 of the rabbit as shown in FIG. 4(C). In such a case, the extensible member 8 is contracted to the position shown in FIG. 4(B) to secure the optimum stretch state and the hook type clasp 2 is then anchored to the loop type clasp 7. After mechanical fastening is thus completed, the hook type clasp 2 and the loop type clasp 7 do not easily come off from each other unless the tab 11 is pulled by considerably strong force.

In the practice of the invention, other fastening mechanisms may be used for the fastening member in place of the mechanical fastener shown in FIG. 3. A typical example of other fastening mechanisms that can be used is a combination of an adhesive tape and a frontal sheet that are designed for fastening. In other words, the adhesive tape and the frontal sheet to which the adhesive tape can be firmly bonded are used in place of the hook type clasp 2 and the loop type clasp 7 shown in FIG. 3, respectively, and are put to the first waist portion 23 (see FIG. 2) of the diaper.

The adhesive tape bonded to the frontal sheet preferably includes a base and an adhesive layer on a lower surface of the base. The distal end of the adhesive tape is bonded to a predetermined portion of the back sheet through its adhesive layer. The distal end of the adhesive tape extending from the bond portion is generally the free end portion and is bonded to a non-release treated surface of the frontal sheet through the adhesive layer of the tap when the diaper is put.

The adhesive tape is preferably rectangular and can take various sizes in accordance with the size of the diaper, desired handling property and other factors. This tape preferably has a width of about 10 to 50 mm and a length of about 30 to 100 mm to secure sufficient bonding strength to the frontal tape and to make fitting and removal of the tape easy.

Examples of the base of the tape include synthetic materials such as polypropylene, polyethylene, propylene-ethylene block polymer, polyester, polyvinyl chloride, polyacetate and polyamide, and natural materials such as cotton, though these examples are not restrictive. These materials may be used either individually or in combination of two or more materials. Particularly, polypropylene, polyethylene, propylene-ethylene block polymer or their combination is suitable as the tape base. To improve adhesion of the adhesive applied for forming the adhesive layer, primer treatment or matt treatment is preferably applied to the lower surface of the tape substrate.

Preferably, the thickness of the base of the adhesive tape is generally within the range of about 10 to 200 μm. When the thickness of the tape base is less than 10 μm, stiffness of the tape is so low that the tape becomes difficult to handle. Further, because mechanical strength is low, the production of the adhesive tape becomes difficult. When the thickness of the tape base exceeds 200 μm, on the other hand, the thickness of the overall tape becomes so great that the tape becomes difficult to handle and creep resistance drops.

The adhesive layer applied to the base of the adhesive tape is generally a synthetic rubber type adhesive or an acrylic adhesive.

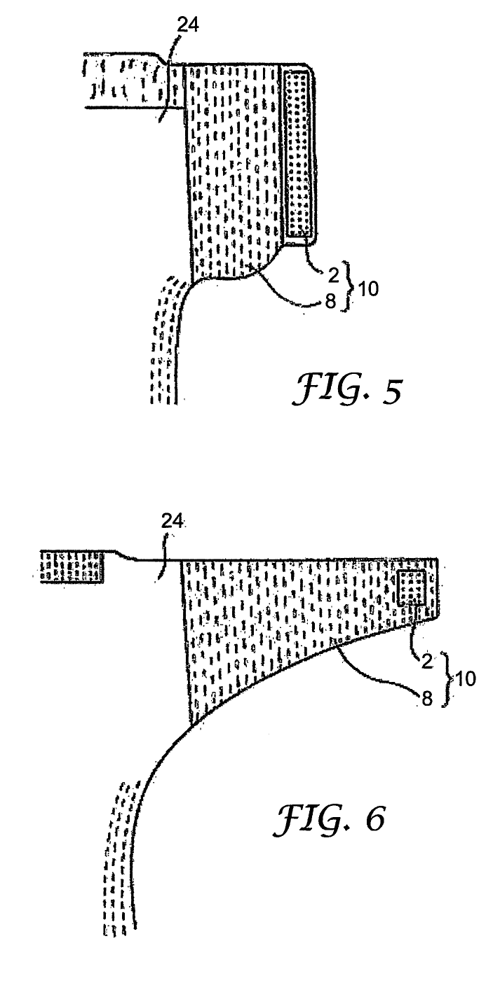

The fastening element according to the invention can be used while fitted to various positions of the disposable diaper in accordance with its construction. As explained with reference to FIGS. 1 to 4, for example, the fastening element 10 can be used as a member of the tab of the disposable diaper 20. In addition, the fastening element 10 can be used as a member of the ear of the disposable diaper as shown in FIG. 5. In the case of the fastening member shown in FIG. 5, the ear is constituted substantially as a whole by use of the extensible member 8 and the hook type clasp 2 is fitted to the side surface of the ear. As shown in FIG. 6, too, the fastening element 10 can be used as a member of the belt of the disposable diaper. In the case of the fastening member shown in the drawing, the belt is constituted substantially as a whole by use of the extensible member 8 and the hook type clasp 2 is fitted to the end portion of the belt.

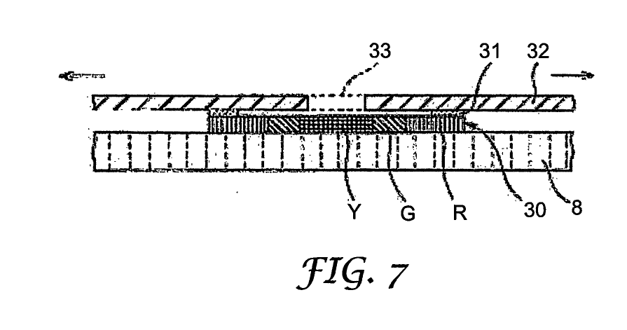

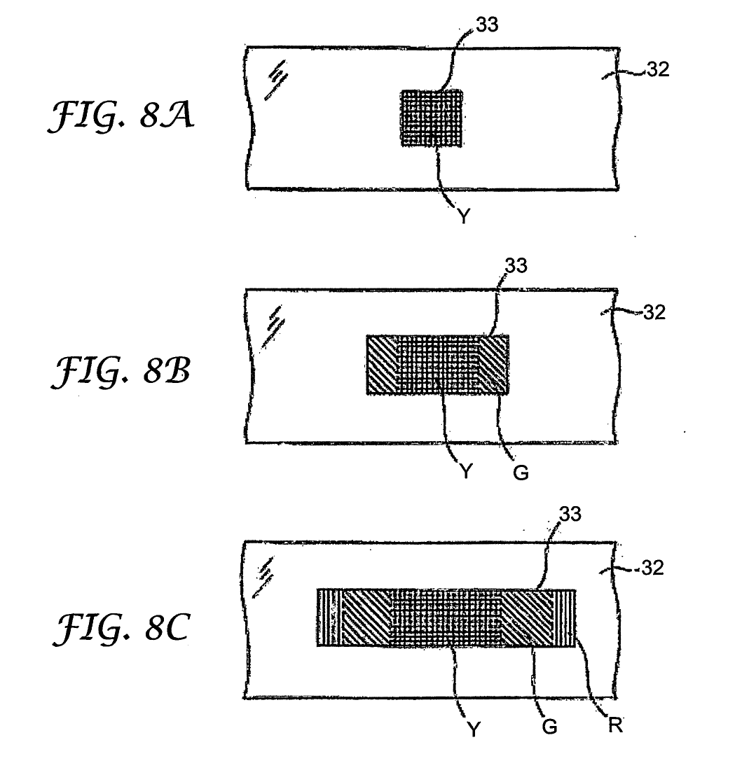

In the fastening element of the invention, the indicator assembled into the extensible member may be an indicator other than the print pattern described above. For example, the indicator of the extensible member is preferably a color chart printed at least partially to the surface of the extensible member. Though the color chart can assume various forms in accordance with the desired recognition effect, it preferably consists of color regions having two or more colors that regularly change in a predetermined direction. In the case of such an indicator, the color chart may be used as such while exposed but is preferably used under the state where it is covered at its substantially center with a cover having a window. FIGS. 7 and 8 are sectional and plan views each showing such an extensible member according to the embodiment of the invention.

In the fastening member 8 shown in FIG. 7, the extensible member 8 fitted to the fastening member (not shown) has the color chart 30 on its surface. The color chart 30 is formed as a yellow region Y, a green region G and a red region R are sequentially printed from the center of an extensible sheet material. The color chart 30 is extensible in accordance with extension and contraction of the extensible member 8. Here, the yellow region Y, the green region G and the red region R contemplate to display an incomplete extensible region, a proper extensible region and an excessive extensible region, respectively. In the example shown in the drawing, a transparent or translucent cover 32 capable of extending and contracting in accordance with extension and contraction of the extensible member 8 is fitted onto the color chart 30 through the adhesive layer 31. A window 33 is formed near the center of the cover 32. This window 33, too, can extend and contract in accordance with extension and contraction of the extensible member 8. The color regions of the color chart 30 positioned below the cover 32 can be observed through the window 33. Arrows in the drawing represent the pulling direction of the extensible member 8.

FIG. 8(A) corresponds to the sectional view of FIG. 7 and shows the observation state of the color chart 30 in the fastening element before use. In the example shown in this drawing, the extensible member 8 is not at all stretched. Therefore, the color chart 30 is not stretched, either, and the yellow region Y can be observed through the window 33.

When the disposable diaper is put, the extensible member 8 is stretched as shown in FIG. 8(B) and the cover 32 is stretched, too. In consequence, the window 33 of the cover 32 becomes greater. When the extensible member 8 is stretched just to the desired size, the green region G can be observed, too, together with the yellow region Y. Exposure of the green region G represents that extension and contraction of the extensible member 8 is conducted suitably. Therefore, extension and contraction is stopped, the hook type clasp is anchored to the fastening member such as the loop type clasp and they are fixed to each other. Though this embodiment employs the three-color color chart, it would be obvious to those skilled in the art that other color charts and color codes can operate as the scale clearly representing a suitable degree of extension and contraction of the extensible member 8.

Incidentally, when the extensible member 8 is excessively stretched, this excessive extension can be known from the exposure of the red region R as shown in FIG. 8(C). In such a case, the extensible member 8 is contracted to the position shown in FIG. 8(B) to secure the optimum extension/contraction state and the hook type clasp may then be anchored to the fastening member such as the loop type clasp.

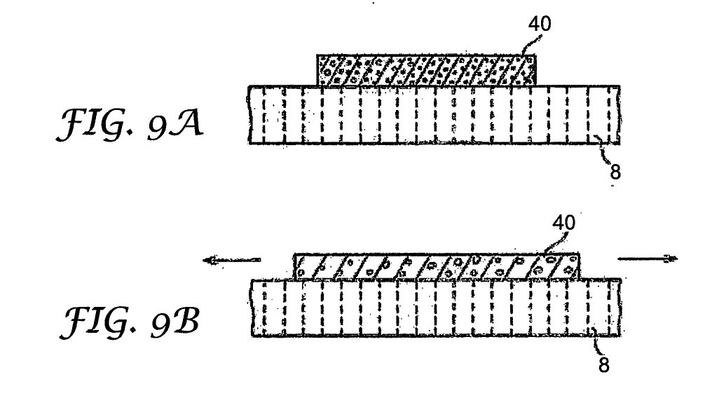

The indicator of the extensible member may be formed of a colored porous sheet applied at least partially to the surface of the extensible member. In such an extensible member, the color of the sheet is variable in accordance with extension and contraction of the extensible member, and the degree of extension and contraction of the extensible member can be easily recognized from the degree of the change of the color. FIG. 9 is a sectional view showing an example of such an extensible member.

Referring to FIG. 9(A), the extensible member 8 fitted to the fastening member (not shown) for forming the fastening element has a porous sheet 40 on its surface. The porous sheet 40 is formed of a colored plastic film and has therein a large number of fine voids. The voids can be formed in the molding stage by blending a filler in the form of particles or wax into the molding material of the film, for example. The shape of the void and its size are not particularly limited in the porous sheet 40.

When the disposable diaper is put, the extensible member 8 is pulled in the direction of the arrows as shown in FIG. 9(B). Then, the porous sheet 40 is stretched, too, in accordance with stretch of the extensible member 8. At this time, the voids contained in the sheet are stretched in the transverse direction and whitening of the sheet occurs with expansion of the voids. In other words, assuming that the porous sheet 40 is colored in blue, the white color increases with stretch of the sheet and reaches substantially the white color through the light blue. According to the invention, the optimum degree of extension and contraction can be easily recognized with eye from the degree of whitening.

1-15. (canceled)

16. A fastening element for detachably fixing a disposable diaper, in which said fastening element is fitted to a tab, an ear or a belt, said fastening element comprises a fastening member having an extensible member, which extensible member has an indicator printed on a face of the extensible member which printed indicator changes with extension and contraction of the extensible member wherein the change in the printed indicator is capable of visually showing a degree of extension and contraction of the extensible member.

17. A fastening element according to claim 16, wherein said printed indicator comprises lines, patterns, numerals, symbols, illustrations, pictures, photographs, color charts, color codes or their combinations.

18. A fastening element according to claim 16, wherein said printed indicator is a print pattern printed at least partially to a surface of said extensible member or inside said extensible member, and said print pattern is deformable in accordance with extension and contraction of said extensible member.

19. A fastening element according to claim 16, wherein said printed indicator comprises a color chart printed at least partially to the surface of said extensible member, and said color chart comprises color regions of two or more colors that regularly change in a predetermined direction.

20. A fastening element according to claim 19, wherein said color chart is covered with a cover having a window-type opening at a substantial center of said color chart.

21. A fastening element according to claim 16, wherein said printed indicator comprises a colored porous sheet applied at least partially to the surface of said extensible member, and the color of said sheet is changeable in accordance with extension and contraction of said extensible member.

22. A fastening element according to claim 16, wherein said fastening member and said extensible member have a unitary structure.

23. A fastening element according to claim 22, wherein said fastening member and said extensible member are unified adjacent to each other.

24. A fastening element according to claim 22, wherein said fastening member is unified with a part of said extensible member.

25. A fastening element according to claim 16, wherein said fastening member is one of the constituent members of a mechanical fastener.

26. A fastening element according to claim 16, wherein said fastening member is an adhesive tape having an adhesive layer.

27. A fastening element according to claim 16, which is fitted to a tab, an ear or a belt of a disposable diaper.

28. An article comprising said fastening element described in claim 16.

29. An article according to claim 28, which is a disposable diaper.

30. A disposable diaper according to claim 29, which is an open type disposable diaper, and said fastening element is fitted to a tab, an ear or a belt of said disposable tab.
